(19) United States
(12) Patent Application Publication
(10) Pub. No.: US 2009/0297437 A1
(43) Pub. Date: Dec. 3, 2009

(54) RADIOACTIVE DEVICE

(75) Inventor: Stephane Lucas, Suarlee (BE)

Correspondence Address: MERCHANT & GOULD PC, P.O. BOX 2903, MINNEAPOLIS, MN, 55402-0903, US

(73) Assignee: FACULTES UNIVERSITAIRES NOTRE-DAME DE LA PAIX, Namur (BE)

(21) Appl. No.: 11/721,959

(22) PCT Filed: Dec. 19, 2005
(86) PCT No.: PCT/BE2005/000185

§ 371 Date: Aug. 13, 2009

(30) Foreign Application Priority Data
Dec. 17, 2004 (EP) 04447284.3

(51) Int. Cl. A61K 51/12; A61P 35/00

(52) U.S. Cl. 424/1.29; 977/906

(57) ABSTRACT

A radioactive or radioactivable nanostructure has a core, the core including at least two atoms, at least one of which being radioactive or radioactivable, and a shell encapsulating the core and selected among a selected material so that at the most, 20% of the radioactive radiation produced by the core are stopped or absorbed by the shell and the manufacturing method thereof.

The various uses of such a nanostructure, and more specifically the use thereof in the medical field, and more specifically in targeted radiotherapy are also disclosed.

### OBJECT OF THE INVENTION

The present invention relates to the assembly of elements, so as to provide radioactive devices.

One of the potential applications involves the injection into the human body, for diagnostic or curative purposes, of such a device, as such, or as a part of a system to be used in curative or diagnostic medicine.

### TECHNOLOGICAL BACKGROUND

Radionuclides are commonly used in various technological fields, in biology but also in other fields, either as markers or as tracers for medium characterization or diagnosis purposes, or as therapeutic agents in nuclear medicine, and more precisely in radiotherapy.

When used as tracers, including in non biological applications, the issue of the reliability of the results being measured could be raised in some cases, and consequently, thereby, the way such results are to be interpreted regarding the characterization of the medium being studied. Indeed, radionuclides, as other tracer types, such as fluorescent markers, for example, are likely to interact with the surrounding medium in which they are located. Such interactions, essentially when the medium is not well known or controlled, could disturb, or even make erroneous the measurement interpretation.

Even if a number of tracer systems have already been suggested in the past for increasing the reliability of the results being measured, there still remains a real industrial interest for alternative solutions.

Moreover, as previously indicated, radionuclides are also used in nuclear medicine as therapeutic agents and, more precisely, in targeted radiotherapy.

Targeted radiotherapy makes use of biological differences between cancerous tumour-forming cells and healthy cells, so as to selectively deliver radionuclides in such a way that the tumour receives a higher amount of radiation than healthy cells. Thereby, the aim is to bring radionuclides near cancerous cells, so as to deliver the maximum dose therein.

In brachytherapy, this is achieved through physically implanting physical elements (seeds) loaded with radionuclides.

Such elements are usually provided as small sticks, being a few millimetres long and having a diameter lower than a millimetre. They are implanted into the human body through surgery.

On the other hand, in targeted radiotherapy, radionuclides are linked with molecular vectors, i.e. with chemical or biological molecules, whether natural or synthetic, such as antibodies, and more particularly, monoclonal antibodies, or fragments thereof, or even peptides, lipids and saccharides having a known affinity for specific markers (receptors at the cell surface) to some types of cancerous cells.

Solutions comprising such vectors are simply injected into the human body, either directly in the subject tissues, or in the blood stream. Thus, such vectors are going to target a cell marker specifically expressed on tumour cells rather than on healthy cells.

In principle, targeted radiotherapy has the advantage of being able to reach all the sick cells throughout the body, whether visible or not, while brachytherapy only allows for the treatment of well located cells.

Numerous patients are currently under treatment through targeted radiotherapy, more particularly by means of molecular vectors such as Zevalin being an anti-CD20 able to be loaded, through an adapted chemistry, with ⁹⁰Y or ¹¹¹In. It is to be noticed that generally, grafting radionuclides occurs in hospital: radionuclides are purchased from a company and the precursors (vectors) from other companies.

The disadvantages of targeted radiotherapy as such are as follows:

1. For each radionuclide, a particular chemistry is to be developed taking into consideration its chemical affinity to the vector. For example, the TYCO company (Mallinckrodt) recently developed chelators for marking proteins with ⁹⁹Tc.

2. The number of radioactive atoms per molecular vector is very low. Usually, it is possible to link one single radioactive atom per vector. However, radiochemists very recently developed dendrimere type molecule patterns in order to increase the number of atoms being grafted per vector.

3. In the case where large size tumours are to be treated, the problem occurs that both the periphery and the centre of the tumour should be equally efficiently treated.

4. It is not possible to visualize through a conventional method, as magnetic resonance, the biodelivery of “grafted drugs” in the human body or in some organs.

There is therefore a need for a radioactive device, being an alternative to solution of the state of the art that could be used efficiently in targeted radiotherapy.

### AIM OF THE INVENTION

The present invention aims to provide a solution for overcoming the above-mentioned state of the art problems.

More particularly, the present invention provides a solution allowing to increasing the radioactivity of the subject devices, for particular uses either in therapeutic or non therapeutic fields.

In particular, the present invention provides a solution in the field of targeted radiotherapy.

In particular, the present invention provides a solution being compatible with such an application, i.e. which should not be radiotoxic to the human and/or animal body.

In particular, the present invention provides a solution intended for efficiently removing cancerous cells from a patient.

More specifically, the present invention provides a solution adapted for eradicating as many cancerous cells as possible in order to avoid any further risk of relapse or dissemination, while protecting as far as possible healthy cells around said cancerous cells.

In other words, the present invention aims at proposing a solution allowing for cancerous cells to be specifically removable.

The present invention also aims at proposing a sufficiently flexible solution allowing for cancerous cells to be efficiently removed whatever their development stage and whatever the availability thereof in the patient's body, i.e. whatever the location thereof in the patient's body, either in periphery of deep inside.

The present invention also aims at proposing a solution able to be tailored over time.

The present invention also aims at proposing a solution allowing for therapeutic agents to be visualized and therefore, to be located in the body of the patient under treatment.

The present invention also aims at proposing a solution adapted to avoid the opsonization problem that could occur in the case where a molecule, being not produced by the human body, is used as a therapeutic agent.

Finally, the present invention aims at proposing to monitor the biodelivery of grafted biomarkers, and this, by means of adequate monitoring devices.

### DEFINITIONS

“Radioactivity” as used in the present invention is the property of an unstable or radioactive atomic nucleus to spontaneously turn into one or more nuclei of other elements while emitting during such a transformation a radioactive radiation.

The word “radionuclide” as used in the present invention means an atom having an unstable atomic nucleus. It is to be understood that in the present invention, the terms “radionuclide” and “radioactive atom” are equivalent.

More specifically, a radionuclide is defined as being a radioactive atom characterized by its proton (Z) and neutron (A-Z) numbers or by its mass number (A).

A radioisotope is defined as a radioactive isotope of a particular element from the Mendeleev's Table (same proton (Z) number but different mass number (A) and hence different neutron number). For example, ¹²⁵I and ¹³¹I are iodine radioisotopes.

In the present invention, the nanostructure is radioactive or radioactivable. More specifically, at least the core of the nanostructure is radioactive or radioactivable, i.e. it is able to produce a radioactive radiation, at least under some conditions.

The expression “radioactive radiation” includes alpha-type radiations, beta-type radiations and gamma-type radiations and the mixtures thereof.

The expression “alpha-type radiation” or “alpha radiation” means a particle radiation corresponding to a helium nucleus, i.e. 2 protons and 2 neutrons.

Consequently, by extension, the expression “radioactive radiation” also encompasses radiations of heavy particles (neutronic radiations and protonic radiations).

The expression “beta-type radiation” or “beta radiation” means a particle radiation corresponding either to an electron (β− radiation) or a positron (β+ radiation).

The expression “gamma-type radiation” or “gamma radiation” means a wave radiation corresponding to a photon. In this respect, there is a distinction between gamma radiations, corresponding to photons being produced by the atom nuclei, RX radiations, corresponding to photons emitted by atom electrons. However, gamma rays, like X rays, are radiations of an electromagnetic nature.

By extension, in so far as X rays could be produced through radioactivity, radioactive radiations, as used in the present invention, also encompasses X rays.

Similarly, in the present invention, the meaning of radioactive radiations also encompasses Auger's electrons.

A radionuclide is characterized by its “half-life” also referred to as “half-life time”, i.e. the time after which half of an amount of such radionuclide is disintegrated.

The “activity” of a radioactive element at a given moment is defined as the number of disintegrations per second at that moment, otherwise stated, the intensity of the radioactivity thereof. It is expressed in Becquerel units.

It should be noted, according to the invention, a “nanostructure” means an assembly of at least several atoms, having a diameter lower than 1 μm, and preferably ranging from about 0.5 nm to 1 μm. The terms “nanostructure” and “nanocluster” are equivalent.

The expression “type” or “species” means radioactive nuclides of the same chemical nature (same proton Z number) and of the same molecular mass (A) and derivatized products from disintegration (ex: ¹⁰³Pd*→¹⁰³Rh+Gamma+RX, the ¹⁰³Pd* and ¹⁰³Rh representing the same radionuclide type).

It should be noted that the notion of “treatment efficiency” by the various radiations is dependent on a physical amount referred to as LET (Linear Energy Transfer). The latter means the radiation energy loss rate in a material, such as, for example, the human body. It is low for a photonic and beta radiation (4 MeV photon: 0.3 keV/μm; 1 MeV β: 0.12 keV/μm) and very high for an alpha radiation (1 MeV alpha: 50 keV/μm).

### SUMMARY OF THE INVENTION

The present invention relates to a radioactive or radioactivable nanostructure comprising a core, said core comprising at least two atoms, at least one of which being radioactive or radioactivable, and a shell encapsulating said core and selected among a selected material so that at the most, 20% of the radioactive radiation produced by the core are stopped or absorbed by the shell.

Preferably, the core comprises at least two radioactive or radioactivable atoms.

Advantageously, the core could comprise from 2 to 20,000 atoms.

Advantageously, according to the invention, the thickness and the chemical nature of the shell material are selected so that at the most, 20% of the radioactive radiation produced by the core are stopped or absorbed by the shell.

Otherwise stated, this means that according to the invention, the nanostructure shell is developed such that it allows the passage of at least 80% of the radioactive radiation produced by the core that are found in the nanostructure surrounding medium and are therefore able to be used in controlled targeted radiotherapy or in detection.

It could be stated that the nanostructure shell according to this invention is, in some extent, “transparent” to radioactive radiations.

More particularly, the nanostructure shell according to this invention could be selected so as to be “transparent” to wave radiation the energy of which ranges from 10⁻² eV to 10⁷ eV. Such a wave radiation could be caused by the disintegration of a core atom (in versus out) or from the environment outside the nanostructure.

However, the shell design (the composition and the thickness thereof) is such that it prevents as far as possible chemical exchanges between the inside of the nanostructure (internal cavity) and the external environment (chemical tightness).

The shell should therefore be considered as a selective barrier useful for setting the exchanges between the inside of the nanostructure and the environment thereof.

For this reason, the size of the nanostructure shell is limited.

The nanostructure shell according to the invention advantageously has a thickness lower than 50 nm and preferably lower than 20 nm.

Such a thickness could, depending on the cases, be achieved either through structuring the shell either as a monolayer, or as several layers, more particularly, advantageously, as three layers.

Advantageously, the nanostructure shell consists in a biocompatible material, i.e. being tolerated by the animal or human organism (a material being non toxic and stable to the endoplasm reticulum).

Preferably, the shell essentially comprises a material selected from the group consisting in amorphous carbon or graphite, metals and the derivatives thereof and polymers, and the mixtures thereof.

Preferably, the shell consists in a material selected from the group consisting in amorphous carbon or graphite, metals and the derivatives thereof and polymers, and the mixtures thereof.

The shell could therefore comprise aluminium and/or titanium oxides.

It should be understood that according to the invention, within the nanostructure, the nanostructure shell surrounds or bounds an internal cavity. Otherwise stated still, the shell “coats” or “encapsulates” the nanostructure radioactive core.

In other words, the internal cavity corresponds to said core.

Otherwise stated, the shell “coats” or “encapsulates” the core.

Preferably, the nanostructure core has a radius lower than 1 μm.

Preferably, the nanostructure core has a radius ranging from about 0.5 nm to about 950 nm, and more preferably from about 0.5 nm to 500 nm, and most preferably from about 0.5 nm to 100 nm, and preferably, from about 0.5 nm to 20 nm, and preferably, from about 2 nm to 20 nm.

Preferably, the nanostructure core has a diameter lower than 1 μm.

Preferably, the nanostructure core has a diameter ranging from about 0.5 nm to about 950 nm, and more preferably from about 0.5 nm to 500 nm, and most preferably from about 0.5 nm to 100 nm, and preferably, from about 0.5 nm to 20 nm, and preferably, from about 2 nm to 20 nm.

Preferably, the nanostructure has a radius lower than 1 μm.

Preferably, the nanostructure has a radius ranging from about 0.5 nm and about 950 nm, and more preferably from about 0.5 nm and 500 nm, and most preferably from about 0.5 nm and 100 nm, and preferably, from about 0.5 nm and 20 nm, and preferably, from about 2 nm and 20 nm.

Preferably, the nanostructure has a diameter lower than 1 μm.

Preferably, the nanostructure has a diameter ranging from about 0.5 nm to about 950 nm, and more preferably from about 0.5 nm to 500 nm, and most preferably from about 0.5 nm to 100 nm, and preferably, from about 0.5 nm to 20 nm, and preferably, from about 2 nm to 20 nm.

It is to be noticed that in size, compared to the shell thickness, the radioactive core thickness is significantly higher.

Preferably, the thickness of the radioactive core accounts, in volume, for at least 60%, and preferably at least 70%, and more preferably at least 80%, and most preferably at least 90%, of the nanostructure.

It is to be noticed that in size, compared to the shell, the radioactive core occupies the major part of the nanostructure volume.

Preferably, the radioactive core accounts, in volume, for at least 60%, and preferably at least 70%, and more preferably at least 80%, and most preferably at least 90%, of the nanostructure.

According to a first embodiment, the core atoms are of the same type, i.e. they have the same atomic number Z, defined as the proton (or electron) number and the same mass number A, defined as the nucleon total number, i.e. the proton and neutron sum.

According to a second embodiment of this invention, the core atoms are different, i.e. their atomic number Z and/or their mass number A are different.

The radioactive radiation produced by the core is selected from the group consisting in alpha radiations, beta radiations, gamma radiations, X rays, Auger's electrons.

Preferably, the radioactive radiation produced by the core is selected from the group consisting in alpha radiations, beta radiations and gamma radiations.

The core atoms could also produce an identical radiation type, but with different energies.

Advantageously, the different core atom types are selected in such a way that they have different half-life times.

Preferably, the radioactive or radioactivable atoms are selected from the group consisted of following radionuclides: ¹⁸F, ⁹⁰Y, ¹⁹²Ir, ¹⁹⁴Ir, ¹⁴²Pr, ¹⁸⁸Re, ³²P, ¹⁶⁶Ho, ⁸⁹Sr, ¹²³Sn, ¹⁴⁹Pm, ¹⁶⁵Dy, ⁷³Ga, ¹⁰⁹Pd, ¹¹⁰Ag, ¹¹¹Ag, ¹¹²Ag, ¹¹³Ag, ¹⁸⁶Re, ¹⁷⁰Tm, ¹⁹⁸Au, ¹⁴³Pr, ¹⁷³Tm, ¹⁵⁹Gd, ¹⁵³Gd, ¹⁵³Sm, ¹⁹⁷Pt, ⁷⁷As, ¹⁶¹Tb, ¹³¹I, ^{114m}In, ¹⁴¹Ce, ^{195m}Pt, ⁴⁷SC, ⁶⁷Cu, ⁶⁴Cu, ^{17m}Sn, ¹⁰⁵Rh, ¹⁷⁷Lu, ¹¹³Sn, ^{113m}In, ¹⁷⁵Yb, ¹⁶⁷Tm, ¹²¹Sn, ¹⁹⁹Au, ¹⁶⁹Yb, ¹⁰³Ru, ¹⁶⁹Er, ³³P, ^{87m}Sr, ¹⁹⁷Hg, ¹⁹⁵Au, ¹⁰³Pd, ²⁰¹Tl, ⁶⁷Ga, ^{103m}Rh, ¹¹¹In, ¹³⁹Ce, ¹¹⁷Sb, ¹⁶¹Ho, ¹²³I, ¹²⁴I, ¹¹⁹Sb, ^{189m}Os, ¹⁴⁹Eu, ¹²⁵I, ⁹⁷Ru, ⁷⁵Se, ¹³⁴Ce, ¹³¹Cs, ⁵¹Cr, ⁶⁷Ga, ⁷³Ga, ⁷⁵Sc, ⁹⁷Ru, ¹⁰³Ru, ¹¹³Sn, ¹¹⁷Sb, ¹²³Sn, ¹³¹Cs, ¹³⁹Ce, ¹⁴¹Ce, ¹⁴⁹Eu, ¹⁶⁷Tm, ¹⁷⁰Tm, ¹⁹⁷Pt, ^{197m}Hg, ¹¹²Pd, ⁵⁵Co, ⁶⁰Co, ⁹⁹Mo, ⁶³Ni, ⁹⁹Tc, ¹⁴C, ³⁵S, ²¹¹At, ⁶⁸Gr, ²⁴¹Am, ¹⁸¹W, ¹³¹Cs, ¹³³Xe, ²¹⁶Bi.

Preferably, the radioactive atoms are selected from the radionuclide group consisted of following radionuclides: ¹⁴C, ³²P, ³³P, ³⁵S, ³⁶Cl, ⁵¹Cr, ⁵⁵Co, ⁶⁰Co, ⁶³Ni, ⁶⁴Cu, ⁶⁷Cu, ⁶⁸Ge, ⁹⁰Y, ⁸⁹Zr, ⁹⁹MO, ^{99/99m}Tc, ¹⁰³Pd, ¹¹²Pd, ¹¹⁰Ag, ¹¹¹Ag, ¹¹²Ag, ¹¹³Ag, ¹¹¹In, ¹²³I, ¹²⁴I, ¹²⁵I, ¹³¹I, ¹³³Xe, ¹³¹Cs, ¹³⁷Cs, ¹⁴²Pm, ¹⁵³Gd, ¹⁵⁹Gd, ¹⁶⁶Ho, ¹⁶⁹Yb, ¹⁸¹W, ¹⁸⁶Re, ¹⁸⁸Re, ¹⁹²Ir, ¹⁹⁴Ir, ¹⁹⁸Au, ¹⁹⁹Au, ²¹⁶Bi, ²¹¹At, ²⁴¹Am.

Preferably, the radioactive atoms are selected from the radio-element group consisted of Pd, Ga, In, Cu, Y, P, Au, I, Lu, Re, At, Bi, W, Tc.

Other radionuclide/radio-element types could also be selected, providing they are compatible with the nanostructure application being provided.

In the present invention, the core or the shell or the nanostructure could further comprise at least one element for imaging corresponding to a contrast medium.

Preferably, the contrast medium is selected amongst elements having a very high electronic magnetic moment selected, for example, from transition metals (Z ranging from 21 to 30, from 39 to 48, from 72 to 80, from 104 to 109), lanthanides (Z ranging from 57 to 71) and actinides (Z ranging from 89 to 103) as well as some elements belonging to the non metals amongst the following atomic numbers: 13, 31, 32, 49, 50, 51, 81, 82, 83, 84. Examples: Cr, Mn, Mg, Fe, Gd, Dy.

In particular, the contrast medium could be selected amongst gallium based alloys, transition metals, actinides, iron oxides and the derivatives thereof.

Such a contrast medium could be chemically grafted on the shell (shell molecules) or physically, for example, through adsorption.

It is to be noticed that the nanostructure could further comprise a targeting agent, preferably located at the shell level.

Even if other targeting agent types in invention application fields other than biology could also be contemplated, the notion of “targeting agent” refers, in the biology field, to an agent able to direct the nanostructure towards some specific targets within the patient, either target-cells, or inside the cell towards target-intracellular compartments.

For example, the targeting agent could be an antibody, and in particular, a monoclonal antibody, or a peptide, or any other protein type known to the man of the art. It could also be a lipid or a nucleic acid.

The antibody could more particularly be an antibody targeting at least a target molecule involved in the angiogenesis, preferably a receptor to VEGF, integrin αvβ3, endoglin (CD105) or annexin Al.

Advantageously, the nanostructure shell is at least partially functionalized by one or more (chemical) functionalization groups, such as OH, NH₂, COOH, SH, . . . well known to the man of the art, with a view to linking said shell to one or more molecules.

Thus, it could be contemplated linking/grafting the targeting agent to the shell through one or more functionalization groups.

Moreover, the nanostructure advantageously has a solid form even if the internal cavity could contain one or more gases such as xenon for instance.

The shell could have an amorphous form or a crystalline form or both.

As far as the intermolecular interactions at the level of the nanostructure are concerned, it is to be noticed that, preferably according to this invention the different nanostructure core atoms could interact with each other via non covalent links, such as links of the ionic, metallic, electrostatic type, or Van der Vaals links, or hydrogen.

Furthermore, it should be stated that preferably according to this invention, the different nanostructure core atoms could interact with the shell (the shell molecules) via non covalent links, such as links of the ionic, metallic, electrostatic type, or Van der Vaals links, or hydrogen.

The latter interaction type could also be of the covalent link type.

Another aim of the present invention relates to the nanostructure such as described for use as a therapeutic agent.

The present invention also relates to the nanostructure to be used as an anti-tumour agent, and more specifically, to be used as an anticancer agent.

The present invention also encompasses the use of the nanostructure as a diagnosis agent.

Another aim of the present invention relates to the nanostructure for treating or preventing tumours, such as cancer tumours, including metastasized cancers.

The present invention is adapted to targeted radiotherapy but precludes brachytherapy as such as defined hereinabove.

This invention also relates to a pharmaceutical composition comprising the nanostructure according to this invention and a pharmaceutically adequate excipient or carrier.

This invention also encompasses the use of the nanostructure and/or such a pharmaceutical composition for manufacturing a drug intended for treating and/or preventing tumour diseases, such as cancers.

This invention also relates to a method for therapeutically treating a disease in a patient comprising administrating the nanostructure or the pharmaceutical composition according to this invention.

Preferably, said method comprises the following steps of:
setting up a preliminary diagnosis comprising listing characteristics of said disease, and more specifically, of the evolution stage in the patient;
estimating the irradiation profile to be achieved in situ in the patient (radioactive radiation type, biodelivery of the dose, intensity, duration, etc.) based on such characteristics;
selecting the nanostructure or the pharmaceutical composition as a function of this profile, comprising selecting the adequate shell type as well as the number and the type of radioactive atoms to be used in the core, the number and the type of targeting agents to be used, as well as the number and the type of the adequate contrast media;
administrating said nanostructure or said composition; and
monitoring and following-up the patient in the course of the treatment, more particularly using one or more contrast media.

Finally, the invention also relates to a method for manufacturing a nanostructure, comprising the following steps of:
providing the core through synthesis according to a method selected amongst physical methods through material flow generated under vacuum and able to condense on a substrate, and chemical methods, or through co-grinding of the components thereof;
encapsulating said core by a shell by means of ion beams, or of a plasma or through gas pyrolysis, such as pyrolysis of carbon gas; and
collecting the thus obtained nanostructure through dissolution of the substrate in a solvent or through mechanical collecting, such as scraping collecting or through a derivatized method.

Preferably, such a method comprises between the core encapsulating and the obtained nanostructure collecting steps, an additional step, referred to as “functionalization step”, during which the shell is functionalized by one or more chemical groups by nitrogen and/or carbon and/or oxygen atomic beams, or by plasma in a reactive atmosphere, depending on the selected chemical group(s).

### BRIEF DESCRIPTION OF THE DRAWING

FIGS. 1 and 2 describe the deposition of a core with some ¹⁰³Pd atoms on a carbon substrate, under low pressure (FIG. 1) and high pressure (FIG. 2).

FIG. 3 shows the number of ¹⁰³Pd atoms that could be located in the core of its size (expressed in nm).

FIG. 4 illustrates, based on the size of the (non encapsulated shell) core, the activity present (recovered) outside such as a core versus the activity produced inside the core.

### DETAILED DESCRIPTION OF SEVERAL EMBODIMENTS OF THE INVENTION

The nanostructure according to the invention, and more particularly the shell thereof, are developed so as to have the following advantageous characteristics:
they have, relative to the outside environment, constant chemical properties, whatever the encapsulated elements are;
they make it possible to modulate the toxicity linked to the introduction of materials into the human body.

It should be noticed that as the nanostructures are obtained, they could be introduced into the human body in various ways:

1. as such, they could be injected in various locations of the human body, either directly in the tissues or in the blood stream;

2. they could be grafted on biological vectors by means of a chemistry specific to the encapsulating material that could have been functionalized with a view to improving the grafting;

3. they could be incorporated into biocompatible capsules and thereby widen the range of medical devices used in brachytherapy.

Depending on the application where the nanostructures are to be used, different configurations of the encapsulated radioactive elements are contemplated:
Use of one single type of radionuclide for achieving a characteristic radiation. In such a case, one single radionuclide type will be encapsulated (coated);
Use of different radionuclide types for obtaining different radiation types. To this end, radionuclides emitting radiations of different nature are gathered and encapsulated. By varying their concentration within the clusters, a control is then available, adapted to improve a radiation (for example beta), relative to another (for example Auger's). When being used in nuclear medicine, such a configuration allows for combining a type of radiation used for diagnostic purposes (for example ^{99m}Te) and a radiation used for curative purposes (for example ²²⁵As). Thereby, it is possible to follow the real time tumour regression.
Use of several radionuclide types for one same type of radiation, but with different energies. Radionuclides emitting radiations of the same type (X, beta or Auger), but of different energies, are then gathered and encapsulated. By varying the concentration thereof within nanoclusters, a control is available, adapted to improve for example a weakly energetic radiation as compared to a highly energetic radiation. When being used in nuclear medicine, such a configuration allows for scattered millimetric tumours to be efficiently and homogeneously treated. It is worth mentioning for example the association of ⁹⁰Y (beta radiation with a mean energy of 934 keV) and ¹⁹⁹Au (beta radiation with a mean energy of 115 keV).
Use of several types of radionuclides with different relational half-life times. As used in nuclear medicine, a radionuclide with a short half-life time imparts a “boost” to the treatment and could be mixed with a radionuclide with a longer half-life time as a remission inducing treatment. Such a modulation should obviously been studied depending on the radio-toxicity of the cells. By way of example, the ¹⁰³Pd (half-life time of 17 days) associated with the ¹⁸¹W (half-life time of 121 days) could be mentioned.
Use of several material types, including one radionuclide or more radionuclides with contrast media. Such contrast media could be magnetic materials, such as iron, Gd and Ga oxides that allow magnetic resonance imaging (MRI), for allowing nanostructures to be located.

### Preferred Choice of Core Radioactive Materials

The following table shows a non limitative list of some radioactive materials, with a curative purpose.

| | | Radiation | |
|---|---|---|---|
| | | (Useful energy | Half-life |
| Atom | Radionuclide | (keV) | time (J) |
| Pd | ¹⁰³Pd | X (22) | 17 |
| Ga | ⁶⁷Ga | β (35) | 3.26 |
| In | ¹¹¹In | γ (184 + 296) | 2.8 |
| Cu | ⁶⁴Cu, ⁶⁷Cu | β (160) | 12 h, 2.58 |
| Y | ⁹⁰Y | β (934) | 2.67 |
| P | ³²P | β (695) | 14.26 |
| Au | ¹⁹⁹Au | β (115) | 3.14 |
| I | ¹³¹I | β (192) | 8.02 |
| Lu | ¹⁷⁷Lu | β (147) | 6.7 |
| Re | ¹⁸⁶Re, ¹⁸⁸Re | β (334, 778) | 3.72 |
| At | ²¹¹At | α (6790) | 7.2 h |
| Bi | ²¹²Bi, ²¹³Bi | α (8000) | 1 h, 0.8 h |
| W | ¹⁸¹W | X (67) | 121 |

Such a list is however not exhaustive. The man of the art could refer to the document entitled: “Radioimmunotherapy of cancer”, Abrams P. G, Fritzberg A. R. Edt, Marcel Dekker Edition 2000, p. 11, p. 57.

Other radioactive elements with a diagnostic purpose and used in Positron Emission Tomography (PET), Single Photon Emission Computed Tomography (SPECT) could also be used: ¹⁸F, ⁸⁹Zr, ^{99m}Te, ¹¹¹In, ²⁰¹Th, ⁵⁸Co, ⁵⁷Ga, . . . .

The table hereinunder compares, by way of example, the number of radioactive atoms of an identical species that could be positioned in a core for a nanostructure with a 1 nm diameter, depending on is the species.

| | Atomic radius | Atom number in a nanostructure |
|---|---|---|
| Element | (angstroms) | with a 1 nm diameter |
| ⁹⁰Y | 1.81 | 21 |
| ¹¹¹In | 1.62 | 29 |
| ¹⁰³pd | 1.38 | 47 |
| ^{99m}Tc | 1.30 | 57 |

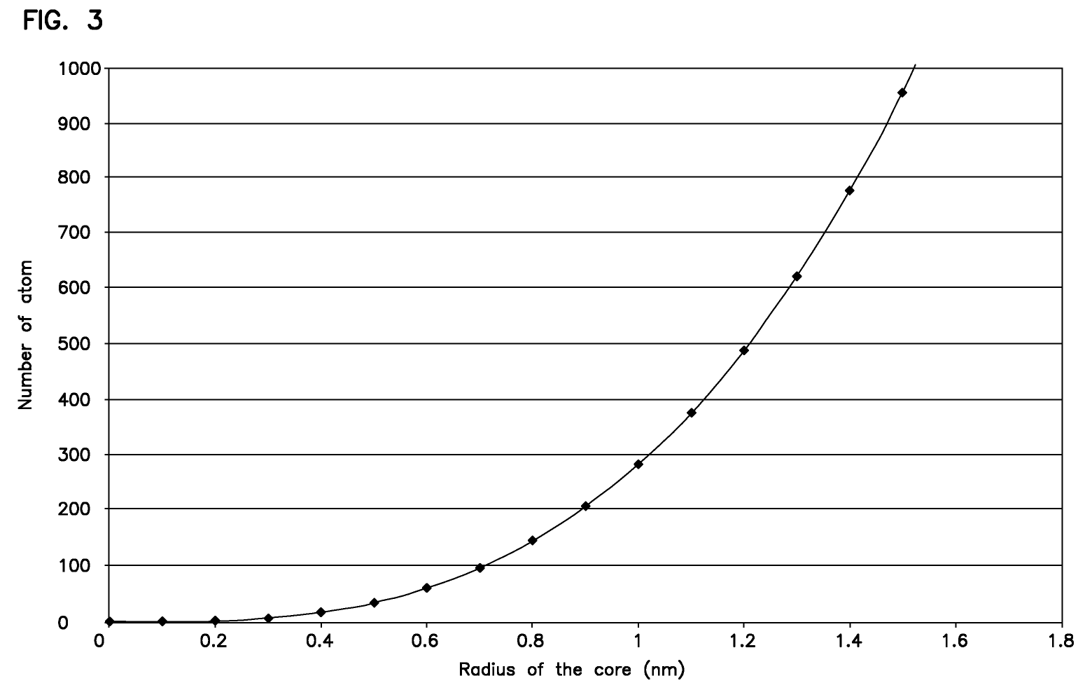

FIG. 3 shows the evolution of the radioactive atom number that could be positioned in a core based on the size of said core, in the particular case where such radioactive atoms are ¹⁰³Pd atoms.

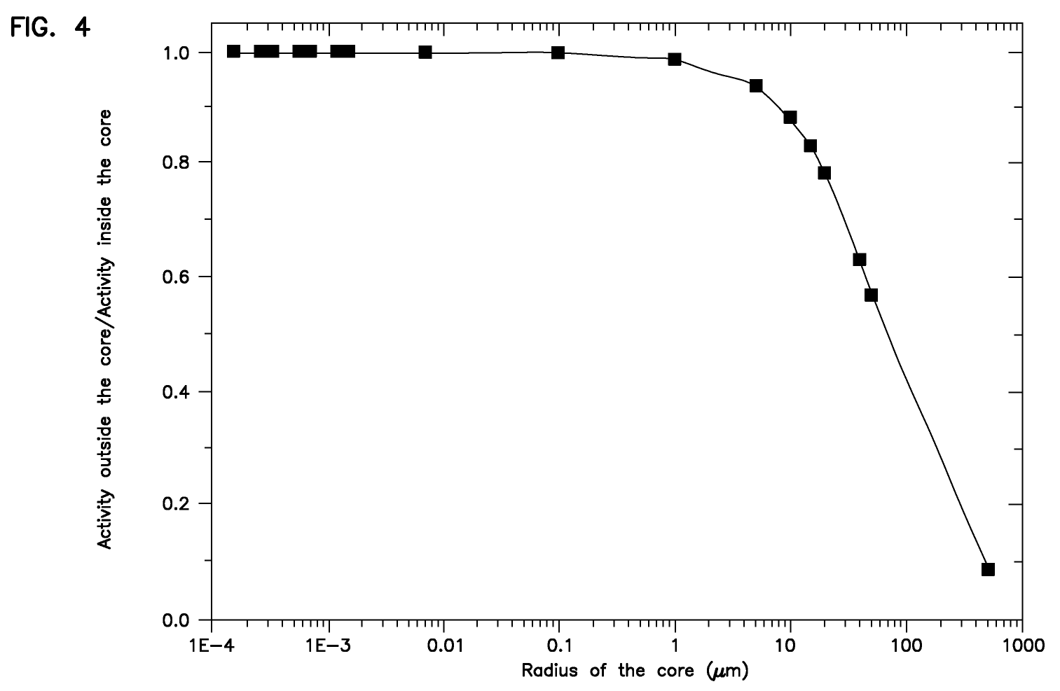

Moreover, FIG. 4 shows, for a core comprising ¹⁰³Pd atoms and non encapsulated with a shell, how the ratio between the activity outside the core and the activity inside the core varies depending on the radius (in μm) of said core.

As illustrated on FIG. 4, it seems that when the core radius is lower than about 1 μm, such a ratio is optimum, as it is relatively constant and equal to 1, meaning that any activity produced by the core goes out into the environment. There is no or little auto-absorption phenomenon or at least such a phenomenon is negligible.

On the other hand, when the core radius is higher than about 1 μm, the activity going out of the core and being in the environment is much lower than the activity produced by the core. It decreases even rapidly as the core radius increases. The auto-absorption phenomenon becomes increasingly more important.

Moreover, complementary results (not shown here) showed that the curve shape does not change if the radionuclide nature in the core should be changed, but that from one radionuclide to another, variations could be observed at the level of the range of core sizes where the auto-absorption phenomenon was sufficiently negligible to be suitable for the intended application.

### Selection of the Encapsulating Material (Shell)

The preferred material for encapsulating (the shell of) the nanostructure will be carbon.

However, other biocompatible materials could also be contemplated: Ta, Ti, Al₂O₃, . . . . As already mentioned hereinabove, (organic and/or inorganic) polymers could also be suitable.

Other materials as described in the literature, for example of the PEG (polyethylene glycol) type, of the PEO (polyethylene oxide) type, poloxomers, polyoxamines, or saccharide derivatives (dextran) could also be contemplated.

Results (not shown here) showed that depending on the chemical nature of the material, the shell size could be selected such that, in some value range, the fraction of radioactive radiation produced by the core able to go across the nanostructure should be optimum, i.e. such that at least 80% of such a radiation go through the nanostructure and should be able to be thereby used, for example, for therapeutic purposes.

It has thus been possible to show that for obtaining a titanium light shell, the size optimum of the shell should be lower than about 50 nm.

Similarly, it has been demonstrated that in the case of carbon, a thin shell having the form of a nanolayer, or even better still of three layers of carbon (diameter: about 6 angstroms), or even four layers, could be suitable.

Depending on the application, the encapsulating material, i.e. the shell material, could be functionalized with groups well known to the man of the art, such as OH, COOH, NH₂, . . . .

Such a functionalization will allow to have it grafted to chemical or biological molecules, but also to make the surface hydrophilic, so as, more specifically, to reduce the opsonization phenomenon, if need be.

Functionalization could also be contemplated for linking the targeting agent such as defined hereinabove to the nanostructure, and more specifically to the shell.

### Radioactive Nanostructure (Nanocluster) Preparation

Preparing such nanostructures occurs in three steps:

1) Core synthesis through physical methods by a flow of vacuum generated material and able to condense on a substrate (PVD, evaporation), co-grinding or via chemical methods (see, for example, M. L. Toebes, J. A. Van Dillen, Journal of Molecular Catalysis A: chemical 173 (2001)75-98).

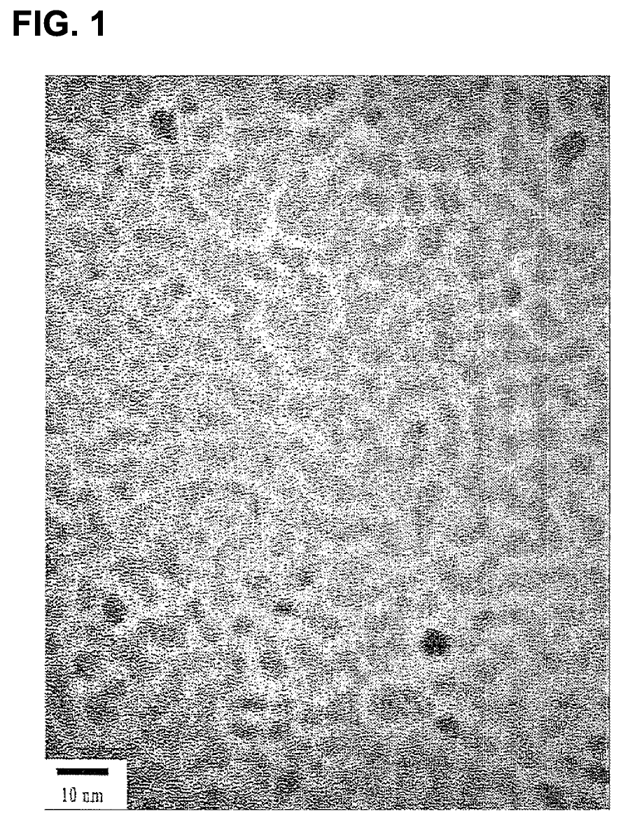

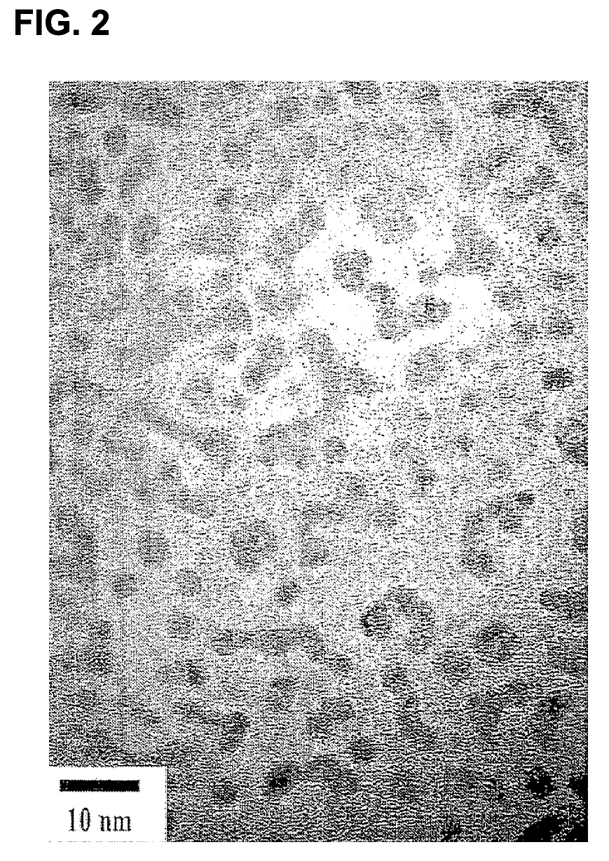

Amongst the above suggested synthesis techniques, the authors have also evaluated the deposition technique through magnetron cathodic spray (PVD) or through evaporation as indicated on FIGS. 1 and 2 and illustrating the preparation of a Pd core (potentially radioactive ¹⁰³Pd) with a mean diameter of 5 nm.

2) Core coating with the shell by means of methods based on ion beams, plasma or even pyrolysis of carbon gas.

3) Functionalization (if needed) of the shell through nitrogen, carbon or oxygen atomic beams or by means of plasma in a reactive atmosphere (N₂, O₂, CF₄, . . . ).

4) Collecting nanostructures via a technique based on:
forming nanostructures and coating on substrates dissolving in a solvent or water (NaCl);
mechanical collection through scraping or a derivatized method;
thermal collection through dripping in a cold solution (10-15° C.) of the substrate coated with nanostructures, which has been brought to a temperature ranging from 100° C. to 200° C.

The above-mentioned techniques are currently used routinely for producing, functionalizing and characterizing nanostructures.

Measuring the number of incorporated radioactive atoms could occur based on the size of nanoclusters and their electronic microscopy imaging, or through atomic force, but also (easier) via the use of radiations emitted by radioactive materials being directly proportional to the number of incorporated atoms.

As previously indicated, incorporating one single radioactive element could be contemplated. However, combining several radioactive elements will be preferred in the scope of this invention.

In a preferred embodiment, a long range radionuclide will be combined (RX or γ emission) with a short range nuclide (β or Auger emission). By way of example, the following couples could be mentioned: ¹⁰³Pd (RX)/⁹⁰Y(β), ¹⁰³Pd(RX)/⁶⁴Cu(β), ¹⁰³Pd(RX)/⁶⁷Ga(β), ¹¹¹In (γ)/⁹⁰Y(β), ⁹⁰Y(β)/²¹¹At(α).

According to another embodiment, radionuclides will be combined emitting the same radiation type, but having different energies: ⁹⁰ T(β)/¹⁹⁹Au(β), ¹⁰³Pd(RX)/¹⁸¹W(RX).

Another configuration would involve combining radionuclides with a diagnostic purpose (^{99m}Tc or ¹⁸F) with radionuclides with a curative purpose (²¹¹At(α)+⁹⁰(β)).

Another configuration would involve combining radionuclides with contrast media (iron oxide, Gd, . . . ).

The present invention further provides the following advantages as compared to the traditional targeted radiotherapy such has proposed so far, including when the shell comprises/consists of carbon:

1. Whatever the type of the grafted radionuclide, a single particular chemistry is necessary, that of the shell material, for example, the chemistry of carbon, being easier to implement.

2. Nanostructures of a few nanometers could contain up to 1,000 atoms. Consequently, on grafting a nanostructure onto a targeting agent, the specific activity is significantly higher to that of current products.

3. In the case of large sized tumours, a radionuclide could be combined in order to treat the outside part of the tumour and another radionuclide with a higher “range” for treating the centre thereof. The same applies for little vascularized tumours (occlusions).

4. Such nanostructures could contain both radionuclides adapted for functional imaging and radionuclides with a therapeutic purpose.

5. In the case where the nanostructure comprises radionuclides with a diagnostic purpose (¹⁸F, ^{99m}Tc, . . . ), the nanostructure biodelivery could be visualized and followed up in the patient's body or in some organs, by means of, for example, a PET or a SPECT camera. Thus, practically, it can be observed that in the case of nanostructures each comprising ten radionuclides with a diagnostic purpose, the signal/noise ratio in the image obtained with a PET or SPECT camera is considerably improved.

6. Another advantage associated to the previous one is that, with a curative aim, by means of the usage of nanostructures both comprising radionuclides with a diagnostic purpose and radionuclides with a therapeutic purpose, it is possible to implement efficient “on-line” internal dosimetry. More specifically, by means of the concurrent (simultaneous) use of those two radionuclide species within one single nanostructure, it is possible to know at any time how many nanostructures are fixed on cancer cells and, thus, to calculate, knowing the number of radioactive atoms in a nanostructure, the dose that such nanostructures are going to locally deliver to cancer cells. This represents a real advantage, in terms of data acquisition speed and reliability of such data over decoupled systems only using either labelled biomarkers in the diagnostic version, or curative purpose biomarkers, as in such a case, it is required for implementing the internal dosimetry of curative purpose biomarkers to use successively over time first systems based on diagnostic purpose biomarkers and a few days later generally subsequently systems based on curative purpose biomarkers (non visualizable by definition).

7. Coupling more radionuclides opens the way to much more performing protocols. For example, based on the characterization of the disease and on the determination of doses to be delivered to the patient by conventional medical techniques, it will be possible to optimize doses to be delivered to sick cells adapting the radiation type and the energy thereof to the size and the distribution of cancer cells, as well as to the localization thereof in the body, or even to combine a high dose flow rate radionuclide (boost) with a low dose flow rate (remission inducing treatment). Thereby, it will be easier to propose treatments in first or second line.

8. Encapsulating metals, either radioactive or not, naturally makes the system reflecting for a sound wave. Echography then becomes possible.

9. Encapsulating contrast media allows for the MRI diagnosis.

10. Encapsulating magnetic components, such as iron or derivatives thereof with radionuclides also makes it possible to combine treatments based on radiation and based on hyperthermia.

1. A radioactive or radioactivable nanostructure comprising a core, said core comprising at least two atoms, at least one of which being radioactive or radioactivable, and a shell encapsulating said core and chosen in a selected material so that at the most, 20% of the radioactive radiation produced by the core are stopped or absorbed by the shell.

2. The nanostructure according to claim 1, wherein the core comprises at least two radioactive or radioactivable atoms.

3. The nanostructure according to claim 1, wherein the thickness and the chemical nature of the shell material are selected so that at the most, 20% of the radiation produced by the core are stopped or absorbed by the shell.

4. The nanostructure according to claim 1, wherein the shell has a thickness lower than 1 μm.

5. The nanostructure according to claim 1, wherein the shell has a thickness lower lower than 20 nm.

6. The nanostructure according to claim 1 wherein the shell is made of a biocompatible material, tolerated by the animal or the human organism.

7. The nanostructure according to claim 1, wherein the shell a material selected from the group consisting of amorphous carbon or graphite, metals and derivatives thereof and polymers, and mixtures thereof.

8. The nanostructure according to claim 1, the diameter of which ranges from between about 0.5 nm and 1 μm.

9. The nanostructure according to claim 1, wherein the radioactive radiation produced by the core is selected from the group consisting of alpha radiations, beta radiations, gamma radiations, X rays and Auger electrons.

10. The nanostructure according to claim 1, wherein the radioactive radiation produced by the core is selected from the group consisting of alpha radiations, beta radiations and gamma radiations.

11. The nanostructure according to claim 1, wherein the core atoms are of the same type.

12. The nanostructure according to claim 1, wherein the core atoms are of different types.

13. The nanostructure according to claim 1, wherein the core radioactive or radioactivable atoms produce radiations of the same type, but with of different energies.

14. The nanostructure according to claim 1 wherein the core radioactive or radioactivable atoms have different half-life times.

15. The nanostructure according to claim 1, wherein the shell is at least partially functionalized by one or more functionalization groups to link said shell to one or more molecules.

16. The nanostructure according to claim 1, wherein the core further comprises at least one imaging element corresponding to a contrasting agent.

17. The nanostructure according to claim 14, wherein the contrasting agent is selected from the group consisting of gallium based alloys, transition metals, lanthanides, actinides, iron oxides and derivatives thereof.

18. The nanostructure according to claim 1, wherein the radioactive atoms are selected from the group consisting of the radioelements ¹⁸F, ⁹⁰Y, ¹⁹²Ir, ¹⁹⁴Ir, ¹⁴²Pr, ¹⁸⁸Re, ³²P, ¹⁶⁶Ho, ⁸⁹Sr, ¹²³Sn, ¹⁴⁹Pm, ¹⁶⁵Dy, ⁷³Ga, ¹⁰⁹Pd, ¹¹⁰Ag, ¹¹¹Ag, ¹¹²Ag, ¹¹³Ag, ¹⁸⁶Re, ¹⁷⁰Tm, ¹⁹⁸Au, ¹⁴³Pr, ¹⁷³Tm, ¹⁵⁹Gd, ¹⁵³Gd, ¹⁵³Sm, ¹⁹⁷Pt, ⁷⁷As, ¹⁶¹Tb, ¹³¹I, ^{114m}In, ¹⁴¹Ce, ^{195m}Pt, ⁴⁷SC, ⁶⁷Cu, ⁶⁴Cu, ^{17m}Sn, ¹⁰⁵Rh, ¹⁷⁷Lu, ¹¹³Sn, ^{113m}In, ¹⁷⁵Yb, ¹⁶⁷Tm, ¹²¹Sn, ¹⁹⁹Au, ¹⁶⁹Yb, ¹⁰³Ru, ¹⁶⁹Er, ³³P, ^{87m}Sr, ¹⁹⁷Hg, ¹⁹⁵Au, ¹⁰³Pd, ²⁰¹Tl, ⁶⁷Ga, ^{103m}Rh, ¹¹¹In, ¹³⁹Ce, ¹¹⁷Sb, ¹⁶¹Ho, ¹²³I, ¹²⁴I, ¹¹⁹Sb, ^{189m}Os, ¹⁴⁹Eu, ¹²⁵I, ⁹⁷Ru, ⁷⁵Se, ¹³⁴Ce, ¹³¹Cs, ⁵¹Cr, ⁶⁷Ga, ⁷³Ga, ⁷⁵Sc, ⁹⁷Ru, ¹⁰³Ru, ¹¹³Sn, ¹¹⁷Sb, ¹²³Sn, ¹³¹Cs, ¹³⁹Ce, ¹⁴¹Ce, ¹⁴⁹Eu, ¹⁶⁷Tm, ¹⁷⁰Tm, ¹⁹⁷Pt, ^{197m}Hg, ¹¹²Pd, ⁵⁵Co, ⁶⁰Co, ⁹⁹Mo, ⁶³Ni, ⁹⁹Tc, ¹⁴C, ³⁵S, ²¹¹At, ⁶⁸Gr, ²⁴¹Am, ¹⁸¹W, ¹³¹Cs, ¹³³Xe, and ²¹⁶Bi.

19. The nanostructure according to claim 1, further comprises a targeting agent located on the shell.

20. The nanostructure according to claim 19, wherein the targeting agent is linked to the shell by one or more functionalization groups.

21. The nanostructure according to claim 20, wherein the targeting agent is selected from the group consisting of proteins, peptides, antibodies, lipids and nucleic acids.

22. The nanostructure according to claim 21, wherein the antibody is an antibody targeting at least one target molecule involved in angiogenesis, comprising a receptor to VEGF, αvβ3 integrin, endoglin (CD105) or annexin Al.

23. The nanostructure according to claim 1 for use as a therapeutic agent.

24. The nanostructure according to claim 1 for use as an anti-tumour agent or as an anti-cancer agent.

25. The nanostructure according to claim 1 for use as a diagnostic tool.

26. The nanostructure according to claim 1 for treating or preventing tumours, such as cancer tumours.

27. A pharmaceutical composition comprising the nanostructure according to claim 1 and a pharmaceutically adequate excipient.

28. The use of a nanostructure and/or a pharmaceutical composition according to claim 1, for the manufacture of a medicament for treating and/or preventing tumour diseases, such as cancers.

29. A therapeutic treatment method applied to the human being, comprising administering the nanostructure according to claim 1.

30. A method for manufacturing a nanostructure according to claim 1, said method comprising the following steps of:
providing the core by synthesis according to a method selected amongst physical methods through a material flow generated under vacuum and able to condense on a substrate, and chemical methods, or through co-grinding of its components thereof;
encapsulating said core into a shell by of ion beams, or by a plasma or through gas pyrolysis of carbonated gases;
collecting the thus obtained nanostructure by dissolution of the substrate in a solvent or by mechanical collecting, such as scraping collecting or by a derivatized method or by steeping.

31. A method according to claim 30, said method comprising between the step of encapsulating the core and the step of collecting the obtained nanostructure, an additional step, referred to as “functionalization step”, during which the shell is functionalized by one or more chemical groups by means of nitrogen and/or carbon and/or oxygen atomic beams, or by plasma in a reactive atmosphere, depending on the selected chemical group(s).

32. A therapeutic treatment method applied to the human being, comprising the pharmaceutical composition according to claim 27.
